(19) United States
(12) Patent Application Publication
(10) Pub. No.: US 2008/0058737 A1
(43) Pub. Date: Mar. 6, 2008

(54) Absorbent articles and wipes comprising lotion

(76) Inventors: Rosa Alejandra Hernandez, Cincinnati, OH (US); Ilonka Bibiana Laviz, Mason, OH (US); Irene Spalazzi, Mason, OH (US); Ronald Bosman Visscher, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/495,298

(22) Filed: Jul. 28, 2006

(51) Int. Cl. A61F 13/15; A61F 13/20

(52) U.S. Cl. 604/358

(57) ABSTRACT

An absorbent article combination comprising an absorbent article and a wipe. The absorbent article can include one or more lotions and the wipe can comprise one or more lotions. The wipe can be individually packaged and attached to an individually packaged absorbent article.

### FIELD OF THE INVENTION

The present invention relates generally to the field of disposable absorbent articles and wipes.

### BACKGROUND OF THE INVENTION

Absorbent articles, such as sanitary napkins, liners, and tampons, are generally used by women to absorb menstrual or other body exudates. Diapers and incontinence articles are used by babies or other incontinent individuals to absorb and contain body exudates, such as urine and/or feces. Often, the use and changing of these articles is messy and unhygienic. The hands or body of the person changing the article can become contaminated due to a failure of the article to contain the exudates or due to exudates that reside on the user's body or garments after removal of the article. In addition, it can be desirable to clean portions of the user's body or garments, which can also lead to contamination of the hands of the person changing the article. Thus, it would be beneficial to have a means for cleaning soiled skin and/or garments while using or changing an absorbent article.

One means for cleaning soiled surfaces is a wipe. Such wipes can include, for example, wet wipes or dry wipes. Wet wipes used for cleansing are often made of a nonwoven material and comprise a liquid cleansing solution. Some wipes contain a fragrance and/or a lotion. Typically, such wet wipes are generally uniform in color and rectangular in shape. Multiple wipes are often packaged together in a common package, but wipes can also be individually packaged.

Because catamenial absorbent articles and adult incontinence pads are used to manage body exudates, the user can be concerned about the cleanliness and health of the body area that is contacted by the exudates. Many absorbent articles can retain the body exudates in contact with the skin for an extended period of time during the use of the article. This increased skin contact can result in a decreased feeling of freshness and/or cleanliness and/or an increase in malodor or the perception of malodor. In addition, the use of absorbent articles can result in residual body exudates remaining on the skin and hair of the user, which can lead to a decrease in skin hygiene.

As such, it would be desirable to provide a combination absorbent article and wipe product. It would also be desirable to provide a combination absorbent article and wipe product that improves the skin hygiene, skin health, skin feeling, and/or skin softness of a user. Additionally, it would be desirable to provide a combination absorbent article and wipe product that decreases the amount of body exudates that remain on the skin and/or hair of a user of the combination. Furthermore, it would be desirable to provide a combination absorbent article and wipe product that improves the ease of cleaning, increases skin comfort, reduces odor, and/or provides a feeling of cleanliness, dryness, and/or freshness to a user.

### SUMMARY OF THE INVENTION

An absorbent article combination comprising an absorbent article including one or more lotions and a pouch joined to the absorbent article, the pouch having a wipe disposed therein, wherein the wipe includes one or more lotions, is provided. The one or more lotions of the wipe can be related, complementary, synergistic, and/or identical to the one or more lotions of the absorbent article. In certain embodiments, the one or more lotions of the wipe can produce the same benefit as the one or more lotions of the absorbent article. The one or more lotions of the wipe and/or the one or more lotions of the absorbent article can each or collectively produce one or more benefits, such as, e.g., improved skin hygiene; improved skin health; reduction in adherence of body exudates to the user's body; improved ease of cleaning; increased skin comfort; odor reduction; emotional benefits, such as, for example, a feeling of cleanliness, dryness, and/or freshness; or combinations thereof. The wipe can be individually packaged, such as, for example, in a pouch. In certain embodiments, an individually packaged wipe can be attached to an individually packaged absorbent article. A package comprising an absorbent article comprising one or more lotions and a wipe comprising one or more lotions is also provided.

### BRIEF DESCRIPTION OF THE DRAWINGS

FIG. 1 is a cross-sectional side view of one embodiment of the present invention.

### DETAILED DESCRIPTION OF THE INVENTION

The present invention relates to wipes, such as, for example, wet wipes, that can include one or more lotions. The wipes can be used in conjunction with an absorbent article that can include one or more lotions. In certain embodiments, the lotion can improve skin hygiene and/or skin health, such as, for example, by reducing the adherence of body exudates to the user's body; improve the ease of cleaning; increase skin comfort; reduce odor; and/or provide a feeling of cleanliness, dryness, and/or freshness to a user. The lotion can reduce panty and/or other garment soiling and/or leakage. The wipe including one or more lotions can be individually packaged. The individually packaged wipe can be joined to an individually packaged absorbent article. In certain embodiments, the absorbent article can include one or more lotions.

The combination of a wipe with an absorbent article can provide one or more of several advantages. For example, the wipe can provide for cleansing, which can provide the wearer with increased comfort, physically and/or emotionally. The use of the wipe can also provide a reduction in the odor and/or perceived odor associated with menstruation or other bodily exudates. In addition, the use of the wipe can provide emotional benefits to the user by providing a feeling of freshness and/or cleanliness. The addition of at least one lotion to the wipe and/or absorbent article can enhance such advantages by allowing for increased ease of cleaning, such as, for example, by reducing the adherence of any body exudates to the user's skin and hair, by moisturizing and/or soothing the skin, and/or by the incorporation of odor control ingredients into the lotion. Furthermore, in certain embodiments, the one or more lotions of the absorbent article can be transferable to the user's skin and hair by normal contact, user motion, and/or body heat. The one or more lotions of the wipe can be transferable to the user's skin and hair when the wipe is used. Thus, the transferred lotion from the absorbent article can work together with the one or more lotions of the wipe to produce a related, complementary, synergistic, and/or extended effect. In certain embodiments, the lotion on the skin can create a barrier that is hydrophilic. The lotion can reduce the exudates that stay on the skin and can assist the exudates in traveling into the absorbent article, which can reduce leakage.

As used herein, the term “absorbent article” refers to devices that absorb and/or contain a substance, such as, for example, body exudates. A typical absorbent article can be placed against or in proximity to the body of the wearer to absorb and contain various exudates discharged from the body.

“Individually packaged,” as used herein, means that only one item (is packaged a particular package.

“Absorbent article combination” as used herein refers to an absorbent article with a wipe joined thereto.

As used herein the term “joined” refers to the condition where a first member or component is attached, affixed, or otherwise physically connected to a second member or component either directly or indirectly. An example of an indirectly joined member or component might be where the first member or component is affixed or connected to an intermediate member or component that in turn is affixed or connected to the second member or component. The joined relationship between the first member or component and the second member or component can be fixedly or releasably.

As used herein, the term “fixedly joined” refers to a connection that cannot be unattached without at least partially destroying one of the attached components. As used herein, the term “releasably joined” refers to a connection that is meant to be easily released.

As used herein, the term “wipe” refers to a piece of material that can be used to clean a user's skin or a surface. The wipe can be made of any suitable material, such as, for example, nonwoven material, paper material, woven material, knitted material, tufted material, stitch-bonded material incorporating binding yarns or filaments, or material felted by wet-milling. Nonwoven material as used herein is “a manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling.” “ISO 9092:1988, Textiles-Nonwovens-Definition” International Organization for Standardization, 1988. As such, the wipe can be a “nonwoven wipe,” that is, a wipe comprising a nonwoven substrate. The wipe can also be a “paper wipe,” that is, a wipe comprising a paper substrate, such as, for example, a wet-laid substrate like toilet tissue or facial tissue.

The term “wet wipe” as used herein refers to a wipe that includes a substrate that is moistened with any fluid prior to packaging.

As used herein, the term “lotion” is a topical preparation that can include any of a variety of solutions, suspensions, mixtures, emulsions, creams, ointments, salves, medicinal products, and so forth intended to provide a benefit to the surface to which it is applied, such as, e.g., the skin or hair of a user. The lotion can be a fluid or partially fluid (i.e., semi-solid) substance.

As used herein, the term “effective amount of a lotion” refers to an amount of a particular lotion that will be effective in providing the intended benefit when applied to the absorbent article and/or wipe.

The term “image” when used with reference to printing includes, for example, any type of mark, figure, picture, identification code, symbol, icon, pattern, or text, such as, e.g., a word, nomenclature, number, sentence, or instruction. An image can also be a line, line segment, curved line, band, arrow, area of coloration, or any other printed indicia having a purpose of providing a signal or guide to the user.

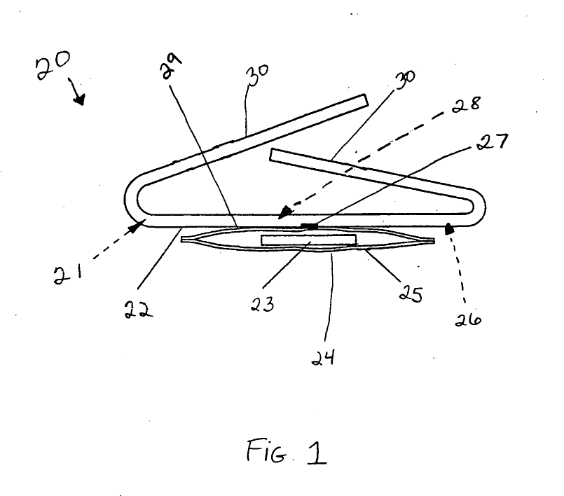

FIG. 1 is a cross-sectional side view showing an absorbent product combination 20 that comprises a sanitary napkin 21 and an individually packaged wipe 23 that is in a pouch 25. In this embodiment, the pouch 25 is joined to the sanitary napkin 21 by a joining means 27. The pouch 25 can be joined to the sanitary napkin 21 either releasably or fixedly. Any suitable means for joining the pouch 25 to the sanitary napkin 21 can be used. One example of a suitable joining means 27 is an adhesive, such as, e.g., a pressure sensitive adhesive. In certain embodiments, the sanitary napkin 21 and pouch 25 are releasably joined using a glue attachment, such as, for example, hot melt glue. The sanitary napkin 21 can have a releasable wrapper 22 that is either releasably joined to or not joined to the sanitary napkin 21. The releasable wrapper 22 can either partially or completely surround the sanitary napkin 21. In certain embodiments, the pouch 25 can be releasably joined to the releasable wrapper 22 of the sanitary napkin. Alternatively, or in addition, the pouch 25 can be fixedly joined.

The sanitary napkin 21 shown in FIG. 1 has a body-facing side 28 and an opposed garment-facing side 26. The body-facing side 28 can comprise a topsheet and the garment-facing side 26 can comprise a liquid impervious backsheet joined to the topsheet. The sanitary napkin 21 can further comprise an absorbent core positioned at least partially between the topsheet and backsheet. The pouch 25 shown has an outwardly facing side 24 and an opposed inwardly facing side 29. The inwardly facing side 29 of the pouch 25 can be joined to the garment facing side 26 of the sanitary napkin 21. FIG. 1 shows the pouch 25 joined to the sanitary napkin 21 in the middle of the garment-facing side 26. The pouch 25 can be joined to the sanitary napkin 21 at any suitable location on the sanitary napkin 21, the releasable wrapper 22, or a package enclosing the sanitary napkin 21. In certain embodiments, the pouch 25 is joined to the sanitary napkin 21 at one end 30 of the napkin 21. Alternatively, the pouch 25 could be located on the body-facing side 28 of the sanitary napkin 21. These locations can permit the pouch 25 containing the wipe 23 to be removed and used before or without opening the sanitary napkin 21.

In certain embodiments, the sanitary napkin 21 can comprise a pair of flaps that each extend from a longitudinal side margin. Suitable sanitary napkins are described in, for example, U.S. Pat. Nos. 5,346,486; 5,009,653; 4,950,264; 4,917,697; 4,687,478; 4,589,876; and 4,285,343. In such embodiments, the flaps of the sanitary napkin 21 are folded over the topsheet in a topsheet facing relationship, and the pouch 25 containing the wipe 23 is releasably affixed to the flaps such that the topsheet facing relationship is maintained. Such an arrangement is described in, for example, U.S. Pat. No. 6,911,022.

Any pouch suitable for holding a wipe can be used. In certain embodiments, the pouch can be separate from the absorbent article prior to attachment. For example, a pouch separate from an absorbent article may not comprise a component of the absorbent article. In other embodiments, the releasable wrapper 22 can comprise a flap (or pouch) to contain a wipe. A suitable flap (or pouch) is described in, for example, U.S. Pat. Nos. 4,556,146 and 5,569,230. The absorbent article can comprise a releasable paper wrapper 22 that surrounds the absorbent article to form an individual package and the pouch comprises a portion of the releasable paper wrapper 22, such as, for example, the pouches described in, e.g., U.S. Pat. No. 5,569,230.

With respect to the sanitary napkin 21 shown in FIG. 1, the sanitary napkin 21 can be individually packaged, such as, for example, in a flexible poly wrapper. In certain embodiments, the releasable paper wrapper 22 of the sanitary napkin 21 can surround the sanitary napkin 21 to form a package. The individually wrapped sanitary napkins can be packaged in a retail package, such as, for example, a box or bag, such as a poly bag.

Although FIG. 1 shows a sanitary napkin 21 as the absorbent article, any suitable absorbent article can be used, such as, e.g., pantiliners, incontinence articles, interlabial pads, or baby diapers. Suitable pantiliners are disclosed in U.S. Pat. No. 4,738,676. Other absorbent articles are described in, for example, U.S. Pat. Nos. 6,059,764; 5,951,536; 5,824,004; 5,702,382; 5,683,375; 5,658,269; 5,439,458; 5,304,161; and 5,300,054.

The absorbent article can further comprise one or more lotions. Any lotion suitable for application to an absorbent article can be used. Suitable lotions include, e.g., lotions that reduce the adherence of body exudates to the user's body; improve the ease of cleaning; increase skin comfort; reduce odor; and/or provide a feeling of cleanliness, dryness, and/or freshness to a user. In certain embodiments, one or more lotions can include lotions that can reduce and/or eliminate menstrual cramps, such as, for example, lotions comprising a soothing aroma or other ingredients.

The lotion can be applied to any suitable location on the absorbent article. In certain embodiments, such as, for example, in the case of an absorbent article comprising a topsheet, such as, e.g., a sanitary napkin, pantiliner, baby diaper, interlabial pad, or incontinence article, one or more lotions can be disposed onto the topsheet of the absorbent article. The lotion can be applied to the absorbent article in any suitable manner, such as, for example,- to the entire surface of the absorbent article or to portions thereof. For example, the lotion can be applied in one or more stripes aligned with the longitudinal centerline of the absorbent article and/or can be applied in a nonuniform manner to the absorbent article. Suitable absorbent articles comprising lotion are described in, for example, U.S. Pat. Applns. 2006/0135920; 2004/0064117; 2003/0206943. Any suitable amount of one or more lotions can be applied to the absorbent article. In certain embodiments, the absorbent article can contain an effective amount of one or more lotions.

In certain embodiments, the wipe 23 comprises one or more lotions. Any lotion suitable for application to a wipe, such as, for example, an aqueous or a non-aqueous lotion can be used. Suitable lotions include, e.g., lotions that reduce the adherence of body exudates to the user's body; improve the ease of cleaning; increase skin comfort; reduce odor; provide a feeling of cleanliness, dryness, and/or freshness to a user; decrease the probability of the soiling of the user's garments; and/or decrease leakage. In certain embodiments, the lotion can increase the cleaning capabilities of the wipe by aiding removal of soiling from the skin. The lotion can also or alternatively provide skin benefits by reducing friction between the skin and the wipe and/or by containing materials that can soothe the skin, such as, for example, aloe or chamomile. Examples of suitable lotions are disclosed in, for example, EP 808151; EP 763341; and WO 00/57843. The wipe 23 can contain substances such as silicones that can help to inhibit body exudates from adhering to the user's body; skin conditioning substances similar to those used in hand lotions, such as, e.g., aloe and Vitamin E; other substances for applying to the skin, such as, e.g., witch hazel; and/or any other substances suitable for inclusion in wipes.

The lotion can be added to the wipe 23 in any suitable manner and at any suitable time, such as, e.g., prior to use by the user. For example, the wipe 23 can be impregnated with the lotion and/or lotion can be applied to one side of the wipe 23. In certain embodiments, a first lotion is applied to the first side of the wipe 23 and a second lotion is applied to the second side of the wipe 23. The lotion can also be included in the wipe 23 separately from any cleansing solution that can be present such as, for example, by applying the lotion to the wipe 23 prior to or after any cleansing solution can be added. In certain embodiments, the one or more lotions can be disposed together or separately on at least a portion of the wipe 23.

In certain embodiments, the one or more lotions included in the wipe 23 can have one or more benefits that are related to, complementary to, synergistic to, or the same as the one or more benefits of the one or more lotions contained on the absorbent article. As used herein, the term “related” means to have characteristics that are logically associated. For example, two different lotions that impart the benefit of odor reduction can be related since both lotions can impart the same benefit.

As used herein, the term “complementary” refers to two or more elements that when detected together by a user can produce a desired effect that is different from the effect of using one element individually. For example, a first lotion that reduces odor and a second lotion that enhances the cleaning ability of a wipe could together produce a feeling of freshness and/or cleanliness.

As used herein, the term “synergistic” refers to elements that when detected together by a user produce a reaction in the user that is stronger or more desirable than the reaction created when the user detects the elements individually. For example, a lotion that reduces the adherence of body exudates to a user's body and a lotion that improves the ease of cleaning could provide an enhanced benefit and/or feeling of freshness and/or cleanliness compared to that provided by either lotion individually.

In certain embodiments, the one or more lotions of the absorbent article and the one or more lotions of the wipe can be the same and/or substantially the same. For example, the absorbent article and the wipe could be considered to include the same or substantially the same lotion if the wipe includes a first lotion and the absorbent article includes a first lotion that further comprises an additional ingredient, such as an immobilizer. In certain embodiments, the one or more lotions of the absorbent article and the one or more lotions of the wipe can be considered the same if the lotions have identical compositions, or even if the compositions differ, they have no readily discernible differences when detected by a typical user.

In certain embodiments, the use of one or more lotions on the wipe 23 and/or absorbent article can result in an extended effect, that is, the effects of the lotion can persist for a longer period of time compared to the use of the lotion on either the wipe 23 or the absorbent article individually. The use of one or more identical lotions on the wipe 23 and/or the absorbent article can also result in a synergistic effect. For example, use of an identical lotion that reduces adherence of body exudates to the user's skin and hair on the wipe 23 and the absorbent article could be more effective than use of the lotion on either the wipe 23 or the absorbent article individually.

The one or more lotions included in the wipe 23 and/or absorbent article can comprise any ingredients suitable for use on a wipe and/or absorbent article. Suitable ingredients include, for example, emollients, immobilizers, skin treatment agents, soothing or protective agents, vegetable extracts, floral extracts, water, viscosity modifiers, fragrances, disinfectant antibacterial actives, antifungals, pharmaceutical actives, film formers, deodorants, opacifiers, astringents, solvents, and/or stabilizers, such as, e.g., cellulose derivatives, proteins, and lecithin.

In certain embodiments, the lotion can comprise at least one emollient. As used herein, an “emollient” is a material that softens, soothes, supples, coats, lubricates, moisturizes, and/or cleanses the skin and hair. Any suitable emollient can be used, such as, for example, a petroleum-based emollient, such as, e.g., mineral oil and/or petrolatum; a fatty acid ester type emollient, such as, e.g., methyl palmitate, methyl stearate, isopropyl laurate, isopropyl myristate, isopropyl palmitate, ethylhexyl palmitate, lauryl lactate, and/or cetyl lactate; an alkyl ethoxylate type emollient, such as, e.g., lauryl, cetyl, and/or stearyl ethoxylates; a fatty acid ester ethoxylate type emollient; a fatty alcohol type emollient, such as, e.g., cetyl alcohol and/or stearyl alcohol; a polysiloxane type emollient, such as, e.g., phenyl-functional polymethylsiloxane compounds such as Dow Corning 556 Cosmetic-Grade Fluid, polyphenylme-thylsiloxane, cetyl or stearyl functionalized. dimethicones such as Dow 2502 and Dow 2503 polysiloxane fluids, and/or compounds disclosed in U.S. Pat. No. 5,059,282; and/or mixtures thereof. Other suitable emollients can include, for example, spermaceti or other waxes; fatty acids; and fatty alcohol ethers, such as stearic acid; propoxylated fatty alcohols; fatty esters of polyhydroxy alcohols; and/or lanolin and its derivatives.

In certain embodiments, the lotion can comprise at least one emollient and at least one immobilizer that can be capable of immobilizing the emollient on the absorbent article to which the lotion composition is applied. Suitable immobilizing agents can include, e.g., C14-C22 fatty alcohols; C16-C18 fatty alcohols, such as, e.g., cetyl alcohol, stearyl alcohol, and mixtures thereof; C14-C22 fatty acids; C16-C18 fatty acids, such as, e.g., palmitic acid, stearic acid, and mixtures thereof; C14-C22 fatty alcohol ethoxylates; polyhydroxy fatty acid esters, such as, e.g., sorbitan palmitate (e.g., SPAN 40), sorbitan stearate (e.g., SPAN 60), sorbitan behenate, glyceryl monostearate, glyceryl monopalmitate, glyceryl monobehenate, sucrose mono- and di-strearate and sucrose mono- and di-laurate; polyhydroxy fatty acid amides; waxes, such as, for example, carnauba, beeswax, catidelilla, paraffin, ceresin, esparto, ouricuri, and rezowax; and mixtures thereof.

In certain embodiments, the lotion can comprise one or more skin treatment agents, such as, for example, agents useful in treating and preventing erythema, malodor, and bacterial skin disorders. Suitable skin treatment agents include, for example, hexamidine, zinc oxide, and niacinamide.

The lotion can be applied to the absorbent article in any suitable manner. In certain embodiments, one or more lotions can be applied to the body facing surface of the absorbent article, such as, for example, the topsheet of a sanitary napkin. Any of a variety of application methods can be used. Suitable methods include spraying, printing (e.g., flexographic printing), coating (e.g., gravure coating), extrusion, or combinations of these application techniques, e.g., spraying the lotion composition on a rotating surface, such as a calender roll, that then transfers the composition to the outer surface of the sanitary napkin topsheet.

The wipe 23 can be a nonwoven wipe comprising a substrate comprising a nonwoven web of natural fibers, synthetic fibers, or mixtures of natural and synthetic fibers wherein the substrate is not considered paper. Suitable natural fibers include, e.g., cellulosic fibers, such as wood pulp fibers, cotton, and rayon. Suitable synthetic fibers include fibers commonly used in textiles, such as, for example, polyolefins, such as polyester and polypropylene fibers. The wipe can be a nonwoven wipe comprising a substrate that comprises viscose rayon and/or wood pulp. Alternatively, the wipe 23 can be a paper wipe, such as, e.g., a wipe comprising a substrate that is made of a wet-laid material in a manner similar to toilet tissue or facial tissue. In certain embodiments, the wipe can comprise silk.

The wipe 23 can be “flushable”, that is, it is of a size of is capable of disintegrating to pieces sufficiently small such that when flushed in a toilet and when being transported in the sewer system, it does not plug any element of a sewer system. Any wrapping material associated with the wipe, such as, for example, the pouch material, can also be flushable. Wrapping materials that meet such criteria can include, for example, water soluble materials, such as, e.g., silicone-treated polyvinyl alcohol films, films coated with a polyvinyl alcohol, and tissue coated or impregnated with polyvinyl alcohol. Suitable materials are disclosed in, e.g., U.S. Pat. No. 4,755,421.

The wipe 23 can be any size suitable for cleansing the skin or providing other benefits when using or changing an absorbent article. In certain embodiments, the wipe can be rectangular. The wipe 23 can be greater than or equal to about 4 square inches (about 25 square centimeters) in size, greater than or equal to about 9 square inches (about 50 square centimeters) in size, less than or equal to about 225 square inches (about 1,450 square centimeters) in size, between about 16 square inches (about 100 square centimeters) and about 50 square inches (about 320 square centimeters), or about 35 square inches (about 225 square centimeters) in size.

The wipe 23 can be a wet wipe that comprises a liquid-based cleansing solution. The wipe 23 can also or alternatively be scented. In certain embodiments, the wipe 23 can comprise a nonwoven fabric impregnated with the cleansing solution. The cleansing solution can include, for example, surfactants, alcohols, fragrance compositions, antimicrobial agents, pH buffers, and combinations thereof. The wipe 23 can contain substances such as silicones that can inhibit body exudates from adhering to the user's body. In certain embodiments, the cleaning solution can contain skin conditioning substances similar to those used in hand lotions, such as, e.g., aloe and Vitamin E; other substances for applying to the skin, such as, e.g., witch hazel; or any other substances known in the art for inclusion in wet wipes.

One or more fragrance compositions can be included in the cleansing solution in order to produce a scented wipe. Alternatively, the one or more fragrance compositions can be included in the wipe 23 separately from any cleansing solution such as, for example, by applying the fragrance composition to the wipe 23 prior to or after any cleansing solution can be added. Another example might be a wipe 23 that includes a separate component such as, for example, a carrier material comprising a fragrance composition. Additionally, the one or more fragrance compositions can be disposed together or separately on at least a portion of the wipe 23.

Other embodiments can include dry wipes or wipes containing non-aqueous cleaning solutions such as, for example, mineral oils, and the emollient described in U.S. Pat. No. 4,481,243.

In certain embodiments, such as, e.g., when the wipe 23 can comprise one or more lotions applied to only one side of the wipe 23. The wipe 23 can be printed to communicate to the user which side of the wipe 23 should be used. For example, one side of the wipe 23 can printed and the other side of the wipe 23 can be free of printing or can be printed with a different design. Because one side of the printed wet wipe 23 is distinct from the other side of the wipe 23, the user can orient the wipe 23 so as to use the proper surface of the wipe 23 to clean the contaminated body areas. Either side of the wipe 23 can be printed on, as long as the communication is clear to the user as to which side of the wipe 23 has the enhanced benefits. Thus, in certain embodiments, the wipe 23 can be printed on the side of the wipe 23 with the enhanced benefits. In certain embodiments, the wipe 23 has a first printing on the side of the wipe 23 with the enhanced benefits and a second printing on the reverse side of the wipe 23. For example, the wipe 23 could be printed with images such as dots or flowers on the side of the wipe 23 with the first lotion and with lines in a pattern on the reverse side or vice versa. In certain embodiments, usage instructions are printed on the side of the wipe 23 with enhanced benefits. In certain embodiments, the wipe 23 is printed such that the user perceives that the indicated portion of the wipe 23 can be one of greater moisturizing, fluid removal, improved cleaning, disinfection, improved softness, or other signals that facilitate an emotional response or proper use of the wipe 23.

In certain embodiments, the wipe 23 comprising one or more lotions can be printed to communicate to the user that the wipe 23 should be used with coordinated absorbent articles. The wipe 23, absorbent article, and exterior packaging can each be printed in a coordinating fashion to communicate that the wipe 23 and absorbent article should be used together, such as, e.g., to allow the user to experience the synergistic benefits of the lotion contained on the absorbent article. For example, the wipe 23, absorbent article, and exterior packaging could each be printed with the same design and/or have printing or coloration in the same shade. In such an arrangement, the printing can be disposed on, for example, the wipe 23, the pouch 25, the absorbent article, the releasable wrapper 22, a package containing the absorbent article, and/or an exterior packaging such as, for example, a bag or a box in which a plurality of absorbent article combinations are packaged. In certain embodiments, the wipe 23, pouch 25, absorbent article, and/or package can be printed with information about the corresponding absorbent article and/or wipe 23.

The wipe 23, pouch 25, or wipe exterior package can also or alternatively be printed with a design that communicates that wipe 23 coordinates with the absorbent article to which it is attached. For example, the wipe 23 can be printed to coordinate and/or correspond to printing and/or coloration on the absorbent article or absorbent article packaging to which the wipe 23 is joined.

In certain embodiments, the wipe 23 can be individually packaged and can be joined to the absorbent article. One individually packaged wipe 23 can be joined to one individually packaged absorbent article, more than one individually packaged wipe 23 can be joined to one individually packaged absorbent article, one individually packaged wipe 23 can be joined to more than one absorbent article that can be individually packaged, more than one wipe packaged together can be joined to one or more individually packaged absorbent articles, one or more individually packaged wipes can be joined to one or more absorbent articles packaged together, or any suitable combination. In certain embodiments, the wipe can be individually packaged, but is not joined to the absorbent article. For example, the wipes can be packaged in individual pouches and a plurality of individual pouches containing wipes can be packaged with absorbent articles in the same exterior packaging or the plurality of individual pouches can be packaged in a different exterior package from that of the absorbent article. The wipes and the absorbent articles can be co-marketed, such as, e.g., designed to be sold and/or used together, such as, for example, for the same purpose. Alternatively, a plurality of wipes can be packaged together in a common package and then packaged with absorbent articles in the same exterior packaging or the plurality of wipes can be packaged together in a common package that is different from that of the absorbent article. In certain embodiments, a plurality of individually packaged wipes are packaged with a plurality of individually packaged absorbent articles wherein the individual package of one or both of the absorbent article or the wipe is provided with an attachment means that allows attachment of the wipe 23 to the absorbent article, such as described in, for example, WO 2005/115295.

In certain embodiments, the wipe 23 can be packaged in a pouch 25. Any suitable pouch 25 for holding a wipe 23 can be used. For example, the pouch can be made of paper and/or other suitable materials, such as, e.g., polyethylene. When the wipe 23 is a wet wipe, any pouch 25 suitable for maintaining the wetness of the wipe 23 can be used. For example, the pouch 25 can comprise at least one barrier material suitable for preventing evaporation of moisture from the wipe 23 can be used. Suitable barrier materials include, for example, laminates with a layer of ethylene vinyl alcohol (EVOH), propylene vinyl alcohol (PVOH), or aluminum sandwiched between polymeric film layers, such as LDPE (low density polyethylene); metallized, such as aluminized plastic films; metal foils; oriented polyethylene terephtalate (PET); PETG (glycol-modified PET); oriented polyamide; aromatic polyamide; or polymeric films like polyethylene films. In certain embodiments, the pouch 25 comprises paper and/or foil. Also, or alternatively, the pouch 25 can be comprise an inner layer comprising polyethylene (PE), an outer layer comprising a copolymer of polyethylene (PE) and polypropylene (PP) and a subjacent intermediate layer comprising polypropylene (PP).

In certain embodiments, one or more sides of the pouch 25 can be sealed to prevent substantial evaporation of the liquid solution from the time the product is manufactured to the time it is used by the consumer. The pouch 25 can be sealed on each side. Any number of sealing mechanisms can be used including, for example, heat and/or pressure seals, ultrasonic seals, glue seals, zippered track sealing systems such as Dow Company's “ZIP LOCKS” or combinations thereof. The pouch 25 can be vapor impermeable. In certain embodiments, the pouch 25 can be hermetically sealed. In addition, the pouch 25 can be lined with foil, or other suitable material to further reduce evaporation.

The pouch 25 can include a means for unsealing (or opening) the pouch 25 in order to access a wipe 23 contained therein. For example, the pouch 25 can have a scored or die cut line of weakness to allow the pouch 25 to be opened easily with a tab, easy peel label or any other opening mechanism; a “Dry-edge” sticker mechanism, such as, e.g., a sticker with a dry edge for grasping; releasable adhesives; releasable heat and/or pressure seals; perforations; rupture seals; tear seals; or the pouch 25 can be made of a relatively easy to tear material such as, for example, an easy to tear film.

Any suitable joining means can be used to attach the pouch 25 and absorbent article of the absorbent article combination. In certain embodiments, the joining means on the outside of the package of the wipe 23 can be selected and applied such that a sufficient degree of joining between the package of the absorbent article and the package of the wipe 23 is generated. Any suitable adhesive can be used to join the absorbent article and the wipe 23, such as, for example, a pressure-sensitive adhesive, hot melt adhesive, cold melt adhesive, or solvent-based adhesives. The adhesive can be applied to the absorbent article and/or wipe 23 as a continuously coated area or as a pattern, such as, e.g., dots, stripes, spirals, or beads. In certain embodiments, the absorbent article and the wipe 23 can be joined by one or more mechanical fasteners. Suitable mechanical fasteners include, for example, hook and loop fasteners.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference, the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article combination comprising:
a. an absorbent article including one or more lotions; and
b. a pouch joined to the absorbent article, the pouch having a wipe disposed therein, wherein the wipe includes one or more lotions.

2. The absorbent article combination of claim 1, wherein at least one of the one or more lotions of the wipe is related to at least one of the one or more lotions of the absorbent article.

3. The absorbent article combination of claim 1, wherein at least one of the one or more lotions of the wipe is complementary to at least one of the one or more lotions of the absorbent article.

4. The absorbent article combination of claim 1, wherein at least one of the one or more lotions of the wipe is synergistic with at least one of the one or more lotions of the absorbent article.

5. The absorbent article combination of claim 1, wherein the one or more lotions of the absorbent article and the one or more lotions of the wipe provide the same benefit.

6. The absorbent article combination of claim 5, wherein the benefit is selected from the group consisting of: improved skin hygiene; improved skin health; reduction in adherence of body exudates to the user's body; improved ease of cleaning; increased skin comfort;
reduced garment soiling; reduced leakage; odor reduction; emotional benefits, or combinations thereof.

7. The absorbent article combination of claim 6, wherein the emotional benefit is a feeling of cleanliness, dryness, and/or freshness to a user.

8. The absorbent article combination of claim 1, wherein the one or more lotions of the absorbent article and the one or more lotions of the wipe provide an extended effect.

9. The absorbent article combination of claim 1, wherein the pouch comprises a periphery, wherein the periphery is closed.

10. The absorbent article combination of claim 1, wherein the wipe comprises a nonwoven material.

11. The absorbent article combination of claim 1, wherein the wipe is a wet wipe.

12. A package comprising:
a. an absorbent article comprising one or more lotions; and
b. a wipe comprising one or more lotions.

13. The combination of claim 12, wherein the wipe is individually packaged.

14. The combination of claim 12, wherein the wipe comprises a nonwoven material.

15. The combination of claim 12, wherein the wipe is a wet wipe.

16. An absorbent article combination comprising:
a. an absorbent article comprising one or more lotions;
b. a releasable wrapper wherein the releasable wrapper at least partially surrounds the absorbent article; and
c. a pouch joined to the releasable wrapper, the pouch having a wipe disposed therein, wherein the wipe comprises one or more lotions.

17. The absorbent article combination of claim 16, wherein the absorbent article is a sanitary napkin.

18. The absorbent article combination of claim 16, wherein the wipe comprises a nonwoven material.

19. The absorbent article combination of claim 16, wherein the wipe is a wet wipe.

20. The absorbent article combination of claim 16, wherein the pouch comprises a periphery, wherein the periphery is closed.
